(19) United States
(12) Patent Application Publication
(10) Pub. No.: US 2022/0111094 A1
(43) Pub. Date: Apr. 14, 2022

(54) PATHOGEN DECONTAMINATION OF PERSONAL PROTECTIVE EQUIPMENT (PPE), FACE FILTERING RESPIRATORY DEVICES (FFR) AND SINGLE USE MEDICAL DEVICES (SUD)

(71) Applicant: Trevor Percival Castor, Arlington, MA (US)

(72) Inventor: Trevor Percival Castor, Arlington, MA (US)

(73) Assignee: Trevor P. Castor, Woburn, MA (US)

(21) Appl. No.: 17/500,908

(22) Filed: Oct. 13, 2021

Related U.S. Application Data
(60) Provisional application No. 63/090,714, filed on Oct. 13, 2020.

(51) Int. Cl. A61L 2/22; A61L 2/18; A61L 2/26; A61L 2/24

(52) U.S. Cl. CPC A61L 2/22; A61L 2/18; A61L 2/26; A61L 2/24; A61L 2202/24; A61L 2202/122; A61L 2202/14; A61L 2202/15; A61L 2202/11

(57) ABSTRACT

The present invention is directed to methods and apparatus for pathogen decontamination of personal protective equipment (PPE), face filtering respiratory devices (FFR) and single use medical devices (SUD) by supercritical fluids, near critical fluids, and critical fluids with or without polar cosolvents. The invention includes a closed processing chamber for containing and processing the PPE, FFR, and SUD by a supercritical fluid, near critical fluid, and critical fluid with or without polar solvents at a specified temperature and pressure for a specified time sufficient to disrupt or inactivate pathogens and viruses on the PPE, FFR, and SUD without damaging the protective equipment so that they may be revitalized for continued use.

### FEDERAL SUPPORT

Research leading to this invention was in part funded with government support awarded by United States Food and Drug Administration (US FDA).

### FIELD OF INVENTION

The present invention is directed to methods and apparatus for decontaminating pathogen decontamination of personal protective equipment (PPE), face filtering respiratory devices (FFR) and single use medical devices (SUD) by supercritical fluids, near critical fluids, and critical fluids with or without polar cosolvents.

### REFERENCES TO OTHER PATENTS

This application discloses a number of improvements and enhancements to the viral inactivation method and apparatus disclosed in U.S. Pat. No. 5,877,005 to Castor et al., which is hereby incorporated by reference in its entirety.

This application discloses a number of improvements and enhancements to viral inactivation method and apparatus disclosed in U.S. Pat. No. 6,465,168 to Castor et al., which is hereby incorporated by reference in its entirety.

This application discloses a number of improvements and enhancements to the method for inactivating viruses for use in vaccines as disclosed in U.S. Pat. No. 7,033,813 to Castor et al., which is hereby incorporated by reference in its entirety.

This application discloses a number of improvements and enhancements to the method for inactivating viruses as disclosed in published U.S. Patent Application No. 2006/0269928 to Castor, which is hereby incorporated by reference in its entirety.

This application discloses a number of improvements and enhancements to the method for inactivating viruses as disclosed in U.S. Provisional Patent Applications Nos. 63/090,701, 63/090,707, 63/090,711 and 63/090,713 to Castor, which are hereby incorporated by reference in their entirety.

### BACKGROUND OF THE INVENTION

The current COVID-19 pandemic is having a significant impact on the morbidity and mortality of infected patients, and is a threat to the health and welfare of US citizens and people around the world. This pandemic is having a significant impact on the economies and social fabric of all societies. As of mid-September 2020, there were >28,800,000 confirmed cases and 921,423 fatalities worldwide, a case fatality rate of ˜3.1%. In the United States, at the same time, there were >6,700,000 confirmed cases and 199,352 fatalities, a case fatality rate of ˜2.9% (Coronavirus Resource Center, Johns Hopkins University). These statistics reflect an approximately a 10× increase in infections over the last 5 months and an approximately 5× in fatalities. These statistics are unfortunately very fluid since the pandemic is ongoing and have as yet not subsided in the US with the only interventions being containment, mitigation and supportive respiratory care. As of mid-October 2021, there are now >240 million confirmed cases and 4.9 million fatalities worldwide, a case fatality rate of ˜2.0%. In the United States, at the same time, there were >44.6 million confirmed cases and 719,000 fatalities, a case fatality rate of ˜1.6% (Coronavirus Resource Center, Johns Hopkins University). There are however now vaccines or antivirals approved specifically to prevent or treat COVID-19.

The most significant barrier to transmission is personal protective equipment (PPE) including masks and shields for front line medical workers and responders; face masks remain the best remedy for preventing transmission and potentially protection of high dose infection. Unfortunately, during the height of the pandemic, there was a significant shortage of PPE that contributed to higher transmission rates and infection rates in medical personnel and first line responders. There still remains shortages of PPEs that would become exaggerated in a second wave of COVID-19 patients, as is currently being experienced in Europe.

The World Health Organization (WHO) reports that the current global stockpile of PPE is insufficient, particularly for medical masks and respirators, and the supply of gowns, goggles, and face shields is now insufficient to satisfy the global demand. Surging global demand is driven not only by the number of COVID-19 cases but also by misinformation, panic buying, and stockpiling has resulted in further shortages of PPE globally. The capacity to expand PPE production is limited, and the current demand for respirators and masks cannot be met, especially if widespread inappropriate use of PPE continues (WHO, 2020). The U.S. Food and Drug Administration (FDA) also recognizes that the need by healthcare providers and personnel for PPEs such as surgical masks and surgical and isolation gowns, may outpace the supply during the COVID-19 outbreak. The FDA is collaborating with manufacturers of PPE to help facilitate mitigation strategies related to the CO VID-19 outbreak (FDA, 2020).

According to the U.S. Center for Disease Control (CDC), an effective decontamination method for filtering face piece respirators (FFR) such as N95 masks should reduce pathogen burden, not harm fit or filtration performance, and should present no residual chemical hazard. The National Institute for Occupational Safety and Health (NIOSH) found that, as of April 2020, ultraviolet germicidal irradiation, vaporous hydrogen peroxide, and moist heat have shown the most promise as potential methods to decontaminate FFRs. On Mar. 29, 2020, the FDA issued the first Emergency Use Authorization (EUA) for a process to decontaminate FFRs.

Conventional sterilization processes used by medical device manufacturers include steam autoclaves, ethylene oxide, hydrogen peroxide gas plasma and gamma irradiation. While steam is efficient and inexpensive, it cannot be used to treat heat-sensitive materials. While ethylene oxide is utilized to sterilize heat-sensitive, radiation-sensitive and moisture-sensitive materials such as those containing plastics and microelectronics, ethylene oxide is toxic and explosive. Gamma irradiation is not applicable to all single use devices (SUDs) and, for the most part, requires central processing to achieve economies-of-scale because of inherent capital and operating costs. The International Atomic Energy Agency (IAEA) recently reported that radiation is an effective and established tool to sterilize PPE except respiratory face masks as it weakens their fibers.

The WHO does not recommend washing, steam sterilization at 134° C., disinfection with bleach/sodium hypochlorite or alcohol, or microwave oven irradiation to the damage to the mask, toxicity, or loss of filtration efficiency: The WHO concluded that both vapor of hydrogen peroxide and ethylene oxide were favorable in some studies but limited by the models of respirators evaluated. The use of UV radiation can be a potential alternative; however, the low penetration power of UV light may not reach inner materials of respirator or penetrate through pleats or folds. The parameters of disinfection by using UVC light is not yet fully standardized for the purpose of reprocessing masks and respirators; this requires a validation procedure to ensure that all surfaces inside and outside masks are reached by the UVC light with appropriate irradiation time.

There thus remain a significant need for decontamination device that could be used for PPEs and FFRs at the point-of-care in hospitals and nursing homes. This device would be very significant in the current pandemic and for future pandemics. The significance of this device is amplified when used for single-use medical devices (SUDs) such as surgical saw blades, laparoscopy scissors, biopsy forceps; umbilical scissors; gas mask; ophthalmic knife; irrigating syringe; and surgical gown (Lewis, 2000) during supply-chain interruptions.

The current COVID-19 pandemic is having a significant impact on the morbidity and mortality of infected patients, and is a threat to the health and welfare of US citizens and people around the world. The World Health Organization (WHO) reports that the current global stockpile of PPE is insufficient, particularly for medical masks and respirators, and the supply of gowns, goggles, and face shields is now insufficient to satisfy the global demand.

### SUMMARY OF THE INVENTION

The present invention is a method and apparatus for decontaminating PPEs, FFRs and SUDs at the point-of-care in hospitals and nursing homes using the inventor's patented and proprietary CFI™ (critical fluid inactivation) technology in order to protect frontline responders and medical personnel.

The present invention is for a safe and economic method and apparatus for the routine inactivation of coronaviruses and other pathogens that may have become associated with personnel protective equipment (PPE) such as N95 respirators and other single use medical devices (SUDs), reusable devices which contact the body and explanted devices. The COVID-19 pandemic of 2020 has created a higher volume use and demand for personnel protective equipment (PPE) that has not been replenished by traditional supply chains in a timely manner. This imbalance has resulted in the infection and death of many front-line workers in the healthcare and other essential industries in the United States.

The lack of sufficient PPE has also contributed to the continued spread of the extremely contagious SARS-CoV-2 virus, the causative agent of COVID-19. There is thus a high need for point-of-care devices that can reliably inactivate viruses and other pathogens in PPEs such as N95 respirators and other single use medical devices (SUDs) without reducing their efficacy in localized healthcare and hospital settings.

In one aspect of the present invention, a method and apparatus reliably inactivates pathogens in PPEs, so that they may be restored without compromising the integrity of the PPEs, so that they may be reused as virtually new devices.

The present invention for the inactivation of viruses on medical devices uses a proprietary low temperature pathogen inactivation technology called CFI™ (critical fluid inactivation), that utilizes SuperFluids™. SuperFluids™ [SFS] are defined as supercritical, critical or near-critical fluids with or without polar co-solvents such as ethanol. These fluids, such as carbon dioxide, nitrous oxide and propane are normally gaseous at ambient conditions of pressure and temperature. When compressed above their critical pressures and critical temperatures, they become dense phase fluids with enhanced thermodynamic properties of solvation, penetration, selection and expansion.

In an embodiment of the invention, SFS is used to penetrate and inflate virion particles. Upon decompression, the rapidly expanding SFS disrupts the over-inflated virion particles which are inactivated as a result of single-point rupture. CFI™ is purely physical and does not require post-processing to remove chemicals such as psoralens, solvents and detergents. This technique is generally applicable to heat-sensitive, radiation-sensitive and humidity-sensitive devices since it operates at moderately low temperatures (below 50° C.) and uses green, environmental-friendly supercritical fluids.

In another embodiment, the invention is a device that establishes CFI™ conditions for inactivating coronaviruses and other pathogens associated with PPEs and SUDs.

In another aspect, the invention encompasses a CFI™ device for PPEs, FFRs and SUDs that can operate following Good Laboratory Practice (GLP) procedures for the inactivation of coronaviruses including SARS-CoV-2 on PPEs such as gowns, masks and face shields.

This invention embodies a safe and economic device for the routine inactivation of coronaviruses and other pathogens that may have become associated with personnel protective equipment (PPE) such as N95 respirators, single use medical devices (SUDs), and reusable devices which contact the body and explanted devices.

These and other features, aspects and advantages of the present teachings will be better understood with reference to the following drawings, description, examples, and appended claims.

### BRIEF DESCRIPTION OF THE DRAWINGS

FIG. 1 shows before-and-after TEM photomicrographs of bacteriophage Virus Φ-6 before-and-after CFI disruption and inactivation;

FIG. 2 shows before-and-after SEM photomicrographs of Saccharomyces cerevisiae (yeast) before-and-after CFI disruption and inactivation;

FIG. 3 is a CFI™ decontamination device prototype of the present invention;

FIG. 4 is a process flow diagram of the SuperFluids™ CFI™ decontamination device prototype of the present invention;

FIG. 5 illustrates the amount of p24 eluted from beads. Virus was eluted from control (black bars) and treated (white bars) beads by incubating them with 1 ml of growth media for 30 minutes at room temperature. The amount of HIV p24 in the eluate was measured in an ELISA. The SCF is shown in the top row of the x-axis label and the cosolvent in the second row. Experiment numbers are given in the bottom row; and

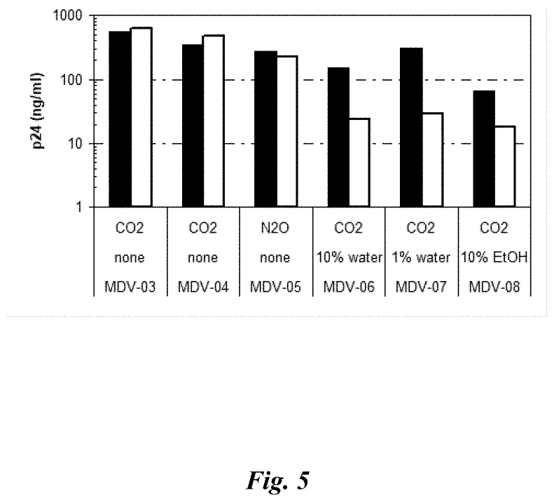

FIG. 6 is a graph illustrating CFI™ of EMC by Freon-22 as a Function of Temperature at 3,000 psig.

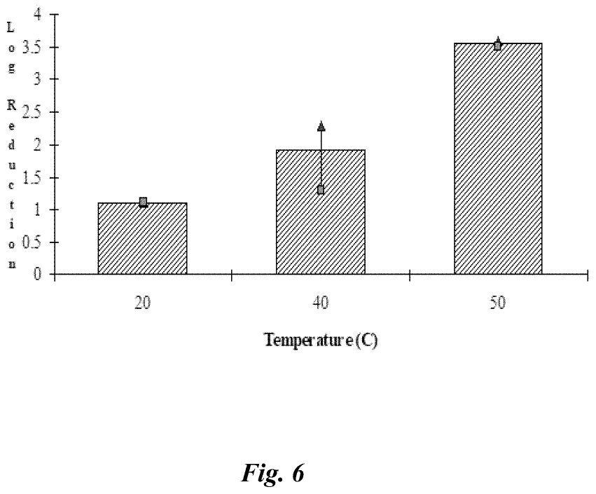

### DETAILED DESCRIPTION OF THE PREFERRED EMBODIMENTS

The present invention is a method and apparatus for decontaminating PPEs, FFRs and SUDs at the point-of-care in hospitals and nursing homes using CFI™ (critical fluid inactivation) technology. CFI™ utilizes supercritical, near-critical or critical fluids such as nitrous oxide and carbon dioxide with or without small molar concentrations of polar cosolvents such as ethanol (referred to as SuperFluids™).

Supercritical fluids, such as carbon dioxide, are normally gases at room temperature and pressure. When compressed, these gases become dense-phase fluids which have enhanced thermodynamic properties of selection, solvation, penetration and expansion. Under these conditions, fluids simultaneously exhibit properties of both liquids and gases—they have the density and solubilization properties of liquids as well as the speed and penetration power of gases. The ultra-low interfacial tension of SuperFluids™ allows facile penetration into nanoporous and microporous structures. As such, SFS can readily penetrate and inflate viral particles. Upon decompression, because of rapid phase conversion, the overfilled particles are ruptured and inactivated.

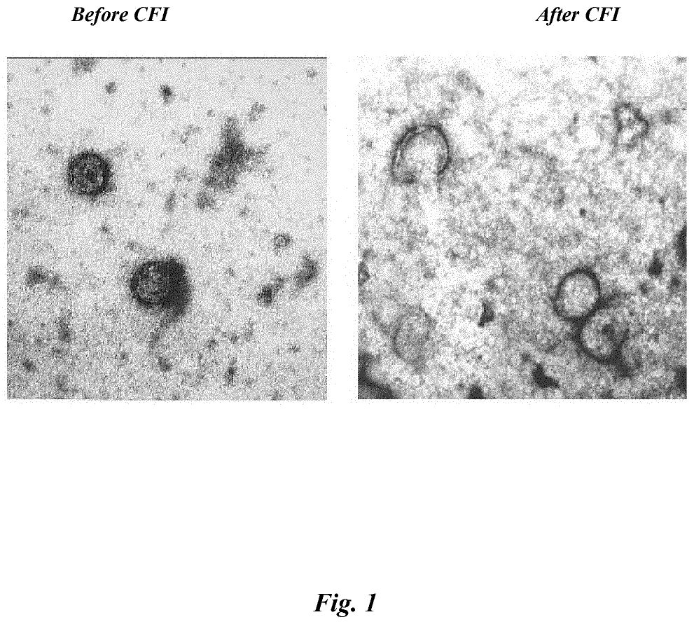

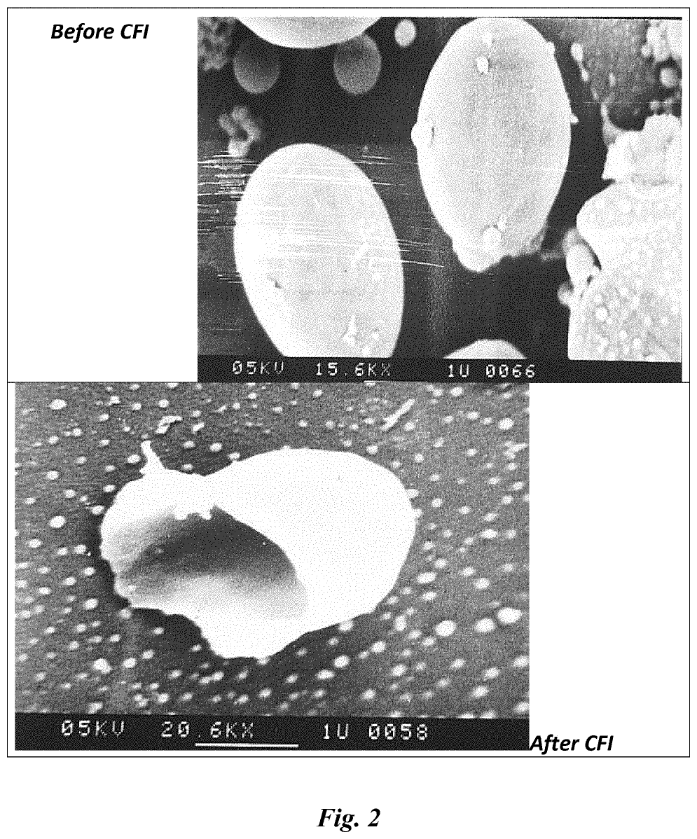

CFI™ pathogen inactivation works, in part, by first permeating and inflating the virus particles with a selected Superfluid™ under pressure. The overfilled particles are then quickly decompressed, and the dense-phase fluid rapidly changes into a gaseous state rupturing the virus particles at their weakest points—very much like the embolic disruption of the ear drums of a scuba diver who surfaces too rapidly. The disruption of viral structure and release of nucleic acids prevents replication and infectivity of the CFI™ treated viral particle. CFI™ has the capability to physically disrupt viral particles as shown by TEM stains of bacteriophage virus Φ-6 and SEMs of the very tough microorganism, Saccharomyces cerevisiae (yeast) before and after CFI™ treatment respectively in FIGS. 1 and 2, illustrating its ability to inactivate both enveloped viruses and a variety of other tough microorganisms.

SuperFluids™ can be used for the gentle and rapid inactivation of both enveloped and non-enveloped viruses without any significant alteration of product quality and biological activity. SuperFluids™ CFI™ (critical fluid inactivation) process inactivates enveloped viruses such as coronaviruses, MuLV, VSV, TGE, BVD, Sindbis and HIV by a lipid solubilization mechanism, similar to the solvent detergent method. SuperFluids™ CFI™ also inactivates non-enveloped viruses surrounded by a tough protein capsid through the physical disruption of viral particles. We have demonstrated CFI™ ability to inactivate non-enveloped viruses such as Hepatitis A, Parvo, Polio, Adeno, Reo and EMC, while preserving biological activity of the treated product. This technology thus makes possible the inactivation of both enveloped and non-enveloped viruses, killing >5-6 logs of virus without any significant alteration of the product.

Competitively, the CFI™ process is effective against both enveloped and non-enveloped viruses, and utilizes low to moderate pressures (1,000 to 3,000 psig) and near-ambient temperatures (20 to 40° C.). Under supercritical fluid conditions, because of ultra-low interfacial tensions, SuperFluids™ will rapidly penetrate the fabric of FFRs to contact and inactivate viruses on the surface and interstitial pores of these medical devices. Additionally, processing times are short and there is negligible impact on product integrity and function. No chemical additives are utilized since the technology is purely physical, and the working fluids are readily separated by gravity from the processed product. The CFI™ process is scalable with low operating costs when compared to ethylene oxide and hydrogen peroxide. Availability of the CFI™ technology in two modules: (i) commercial scale device; and (ii) bench-top device offer versatility that suits diverse customer base. The bench-top pathogen decontamination device is portable and readily deployed to hospitals and healthcare facilities, frontline epidemic zones in remote and highly tropical regions at competitive capital and operating costs.

CFI™ operating design parameters were optimized for the inactivation of different types coronaviruses including SARS-CoV-2 as well as prototypical enveloped and nonenveloped viruses associated with various prototypical PPEs such as N95 and surgical masks. Operating parameters include SuperFluids™ type, cosolvent type and concentration, pressure, temperature, density and polarity as well as residence or contacting times. Initial guidance of operating parameters will, in part, be based on prior preliminary results with HIV-1 on prototypical single use medical devices (SUDs). Final selection of optimum CFI™ conditions is based on experimental data on the inactivation of coronaviruses associated with prototypical medical device materials as well as engineering, economic, environmental, operating, validation and regulatory issues.

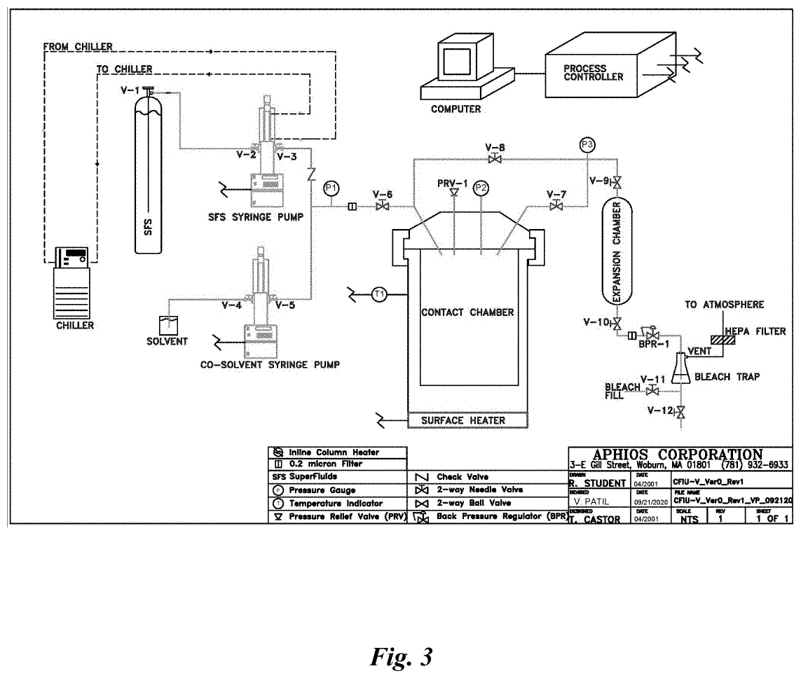

A prototypical CFI™ medical device apparatus, shown as FIG. 3, was designed and constructed. As shown in FIG. 3, this apparatus consists of four primary components: (1) SuperFluids™ delivery system; (2) CFI™ medical device-contacting chamber; (3) expansion chamber; and (4) pressure letdown system. The SuperFluids™ delivery system is a computer-controlled dual pump system, utilizing one syringe pump for neat supercritical fluid and a second syringe pump for water, an alcohol cosolvent or modifier. The pumps are independently controllable, allowing easy and accurate adjustment of the fluid composition.

The CFI™ medical device chamber has a volume of 1 gallon (3.785 liters) with a size of 5.5″ (ID) and 9.75″(depth) and is rated for 3,000 psig (Parr Model 4760). The CFI™ chamber is temperature controlled for stabilizing process operating temperatures. The chamber has isolation valves, V6 and V7, on both ends so that the unit could be readily disconnected. With V6 and V7 closed, a bypass valve, V8, allows for bypassing of the CFI™ chamber during startup and/or shutdown of the virus inactivation process. The expansion chamber is a 20L cylinder, with a pressure rating of 1,000 psig, that allows for a 5:1 rapid expansion of the SuperFluids™ from the CFI™ chamber. The expansion chamber has two isolation valves, V9 and V10, which will allow ready isolation of the expansion chamber for cleaning. The pressure letdown system consists of a back-pressure regulator (BPR-1) for controlled reduction in pressure, a bleach trap for cleansing the exhaust of any residual or carried-over virus, and a vent line connected to a HEPA filter exhaust. The portion of the system in potential direct contact with viruses was placed in a laminar flow hood as the primary virus containment mechanism.

The decontamination device prototype can be run in two modes: dynamic and static. In the static mode, virus-coated N95 face masks and other PPEs were placed in the CFI™ chamber and contacted with the SuperFluids™ for a specific amount of time at a specified pressure and temperature. The CFI™ chamber is be then rapidly exhausted into the expansion chamber. In the dynamic mode, SuperFluids™ is continuously flowed over the virus-coated glass beads at a rate of 20 mL/min, and slowly exhausted at the end of the experiment.

Viral Inactivation Studies Using Masks. Viral inactivation studies were performed to mimic the contamination of masks by pathogens during normal breathing as well as during respiratory illnesses followed by the inactivation treatment. Initially, virus stocks of high enough titers (6 log PFU/mL or TCID₅₀/mL or greater) were used. Known amounts of the virus stocks were sprayed on the masks in 3 sets with replicates of 3-5 for each set. Separate experiments were conducted using masks sprayed with particles of various defined sizes to mimic small aerosol particles (around 1.0 μm diameter) present in the human breath as well as large ballistic droplets present in human coughs and sneezes (I to 1000 μm diameter). Care was taken to keep the amount of the spray constant across all the sets of masks. The first set was a 4° C. control, the second set was a time and temperature (t&T) control, and the last set was subject to different CFI™ inactivation conditions in each experiment. Following the treatment, the treated samples and controls were extracted with the minimal amount of media necessary to allow complete soaking and extraction of the sprayed virus stocks. The residual virus present in each extracted sample was titrated by standard virus titration methods for the respective viruses. The inactivation efficacy of the CFI™ treatment was determined as the reduction Factor (RF), which is the difference in the titers between the controls and the CFI™ treated extractions.

Preliminary experiments were conducted to determine the optimum extraction volumes, as well as cytotoxicity and interference from any materials leaching out of the masks during the extraction procedure. Additional parameters such as the effects of drying of the masks for different durations following the spray procedure were also examined.

SuperFluids™ Type. Carbon dioxide, which has a very modest critical point (31° C. and 1,070 psia), is an excellent candidate since it is inexpensive, non-toxic, non-flammable, and environmentally acceptable. Supercritical carbon dioxide has a density of 0.74 gm/cc at a pressure of 2,000 psia and a temperature of 40° C. At and around these conditions, CO₂ behaves like an organic solvent with solubilization characteristics of a liquid and the permeabilization characteristics of a gas. Carbon dioxide proved quite effective for virus inactivation of HIV viruses associated with medical device components such as N95 masks. In an aqueous media, CO₂ results in the formation of carbonic acid and cause a decrease in pH.

CFI™ virus inactivation experiments were conducted with several different types of SuperFluids™ to evaluate the impact of parameters such as density, polarity and structure on the efficacy of inactivating coronaviruses and other pathogens on medical device components. We investigated the use of carbon dioxide, nitrous oxide, and several fluorocarbons as possible supercritical fluid solvents. With the exception of most fluorocarbons, these fluids all have critical temperatures (T_{c}) near ambient. Nitrous oxide and fluorocarbons have some polarity while carbon dioxide is essentially non-polar. From a practical standpoint, we are particularly interested in using carbon dioxide, which we have shown to be very effective in inactivating enveloped viruses. The thermodynamic properties of CO₂, N₂O and candidate fluorocarbons are listed in Table 1.

TABLE 1
Thermodynamic Properties of Selected SuperFluids ™

| | | Critical | Critical | Dipole |
|---|---|---|---|---|
| | Chemical | Temp. | Press. | Moment |
| Generic Name | Formula | T_{C} (C.) | P_{C} (psig) | (Debye) |
| Carbon Dioxide | CO₂ | 31.1 | 1,055.3 | 0.0 |
| Nitrous Oxide | N₂O | 36.5 | 1,036.3 | 0.2 |
| Freon-22 | CHClF₂ | 96.0 | 707.2 | 1.4 |
| Freon-23 | CHF₃ | 25.9 | 686.5 | 1.6 |
| HCFC-123 | CF₃CHCl₂ | 183.6 | 532.0 | 1.36 |
| HCFC-124 | CHClFCF₃ | 122.2 | 524.5 | 1.47 |
| HCFC-134a | CH₂FCF₃ | 101.1 | 574.2 | 2.06 |

Nitrous Oxide (N₂O) is a small, relatively inert molecule with a polarity of 0.2 Debye. N₂O is an excellent candidate for inactivating non-enveloped viruses by an explosive decompression mechanism because of its density and expansion factor at operating conditions. At 2,000 psig and 22° C., N₂O has a density of 0.93 g/ml and an expansion factor of approximately 500. N20 has also been demonstrated to have minimal or no impact on protein and enzymatic activities over the range of pressures, temperatures and residence or contacting times.

From previous experimental data, Freon-22 (chlorodifluoromethane—CHClF₂) has excellent virucidal properties for both enveloped and non-enveloped viruses. Relative to other chlorofluorocarbons such as Freon-11 and Freon-12 which are being banned by the 1988 Montreal protocol, Freon-22 is very stable and only has a slight ozone depletion potential (ODP of 0.05) because it has a hydrogen atom in its structure. Even though Freon-22 has an ODP that is twenty times less than Freon-11, Freon-22 cannot be used in any new applications after 2010 and in any existing applications after 2020 in accordance with the 1988 Montreal protocol.

Since Freon-22 use and production may be adversely impacted by future environmental concerns, we evaluated alternate refrigerants. Per the listing of thermodynamic properties in Table 1, Freon-23 (trifluoromethane) appears to be an excellent CFI™ candidate because: (i) it is non-chlorinated (the chlorine component of chlorofluorocarbons is thought to be responsible for their negative impact on the ozone layer): (ii) it has a low critical temperature of 25.9° C. (allows operation close to critical conditions while minimizing thermal denaturation of heat sensitive materials); and (iii) it has a relatively large dipole moment of 1.6 Debye (a large potential of solubilizing polar lipids and fats). From a comparison of previous data on the CFI™ virus inactivation of EMC in a FBS matrix, Freon-23 appears to be the best alternate to Freon-22. On the average, Freon-23 inactivated ˜3 logs vs. ˜6 logs of EMC at similar conditions of temperature (50° C.) and pressure (3,000 psig).

Cosolvent Type and Concentration. SuperFluids™ have additional degrees of freedom over a conventional organic solvent in that their solvation capacities can be readily adjusted by changing density (via changes in temperature and/or pressure), and selectivity can be altered by the type and concentration of entrainers or cosolvents. It is possible to enhance the affinity of supercritical CO₂ for phospholipids and fatty acids by adding a “low volatility agent” or cosolvent such as an alcohol. Thus, the ability to use cosolvents is an important feature of the experimental apparatus. Cosolvents such as ethanol were used to enhance the affinity of the supercritical fluids for the extraction of lipids and the penetration of viral particles. These cosolvents are used on the basis of structure, polarity and molecular size. The solvation power of such mixtures can be readily varied by adjustment of pressure, temperature and/or the ratio of supercritical fluid to entrainer. Some experimentation was done on small quantities (around 1 to 10 mole %) of polar entrainers such as water to evaluate their impact on virucidal efficiency.

We also evaluated the impact of a “high volatility cosolvent” such as small quantities of a fluorocarbon such as Freon-23 in CO₂ or N₂O or vice-versa. There are several advantages to such a mixture. While Freon-23 has proven effective for both types of viruses, it has been shown to be particularly effective for enveloped viruses. The latter is supported by our hypothesis that enveloped viruses are inactivated by a phospholipid solubilization mechanism since fluorocarbons have a much greater capacity than N20 or CO₂ to solvate phospholipids. It is conceivable that Freon-23 has demonstrated an ability to inactivate non-enveloped viruses because it can penetrate the protein capsid by first solubilizing protein-interstitial phospholipids and fatty acids. Polio, for example, which is a small viral particle with a very tough protein coat, contains fatty acids between its coat proteins. The highly polar fluorocarbon would improve the efficacy of the mixture to selectively solubilize lipids and fatty acids, more readily allowing the small relatively nonpolar N20 to rapidly penetrate the viral particle. N20, with a larger expansion factor than Freon-23, may be more effective in inactivating nonenveloped viruses. A “high volatility cosolvent” is preferred than a “low volatility agent,” such as water or ethanol, which cannot readily be recycled. In the best of worlds, no cosolvents are utilized.

Pressure. For the most part, pressure was kept constant at around 2,200 in order to evaluate the difference between SuperFluids™ type and cosolvents in the inactivation of HIV. Pressure is, however, a key variable in the SuperFluids™ virus inactivation process. Pressure and pressure drop are important variables because higher pressures are expected to drive more gases into viral particles, and improve the kill efficiency by increasing the disruptive forces during decompression. Inactivation mechanisms are also impacted by SuperFluids™ density, and thus pressure, as well as structure and polarity. Particular attention was paid to the lowest pressure at which the process is effective. Since density and expansion factor approach an asymptote at values about 3 times the critical pressure at near ambient temperature (about 3,000 psig for N20 and CO₂), kill efficiencies would reach a point of diminishing returns around this value. We evaluated the impact of pressure from 1,000 to 3,000 psig.

Temperature. In preliminary studies, all experiments were conducted at or around room temperature in order to maximize the TCID₅₀ of the time and temperature control. Under these conditions, most of the CFI experiments were conducted at sub-critical conditions that may not be optimal for virus inactivation. Temperature will have an impact on both the thermodynamic properties of the SuperFluids™, the configurational structure of the virus, and the integrity of the PPEs and SUDs. Temperature with pressure defines the density of SuperFluids™, and their solvation capacities and expansion factors; the former impacts viral kill by a phospholipid solubilization mechanism and the latter by an explosive decompression mechanism. We have found that N20 is equally effective in inactivating murine leukemia virus (MuLV) at 1,000 psig and 10° C., 2,000 psig and 22° C. and 4,000 psig and 40° C., conditions which provide almost identical densities. CFI™ technology thus favors lower temperatures, which are better for material integrity. Lowering the temperature often requires the lowering of pressure because density is directly (but not linearly) proportional to pressure and inversely proportional to temperature.

We limited our evaluation to temperatures around body temperature, 37° C., within a range of 4° C. to 60° C. Use of temperatures in the higher end of this range has been found to be important in the inactivation of certain viruses. Temperature may also impact the configurational structure of the virus and its penetrability by the SuperFluids™. Poliovirus, for example, undergoes normal structural changes as temperature is increased above ambient, which may make it more susceptible to inactivation. Higher temperatures will also adversely impact the integrity of the medical device.

Density and Polarity. The density of SuperFluids™ is determined by both temperature and pressure. Density will impact kill efficiency through lipid and fatty acid solubilization mechanisms, and explosive decompression mechanisms. Polarity is defined by the SuperFluids™ type, as well as the type and concentration of the polar entrainer. Polarity will impact kill efficiency through permeability enhancement of the viral particle. Both of these parameters were co-evaluated from experiments described above.

Residence or Contacting Time. Sufficient residence or contacting time is necessary for the SuperFluids™ to contact, interact with, and penetrate the viral particle. We have discovered that the SuperFluids™ CFP′ process is mass transfer limited by the ability of the SuperFluids™ to diffuse though the aqueous phase to reach the viral particle. In so doing, we have reduced the contacting time from tens of minutes to tens of seconds by the use of a continuous flow injection technique. Since this technique will not be utilized in the experiments to be conducted, we plan to vary the residence or contacting time to evaluate viral inactivation efficacy. We will also evaluate circulating the SuperFluids™ over the medical device materials to evaluate the impact of circulation on virus kill and residence time required. Residence time will also impact the cycle time for the CFI™ device and its economics. A shorter cycle time will increase throughput of medical devices per unit capital piece of equipment, and impact both depreciated capital and operating costs.

Virus and Cells. We evaluated efficacy against four (4) coronaviruses—two low pathogenicity human coronaviruses, one well-studied mouse coronavirus and the novel coronavirus, SARS CoV-2, the causative aunt of COVID-19 under BSL-3 laboratory conditions. Additionally, other pathogens of interest include a prototypical enveloped virus, Bovine Viral Diarrhea Virus (BVDV) as a surrogate for Hepatitis C; a prototypical nonenveloped virus Human Adeno-2 Virus (HAd-2); a prototypical Gram-negative bacteria, Escherichia co/i; and a prototypical Gram-positive bacteria, Bacillus subtilis.

Coronaviruses. Mildly pathogenic human coronavirus (HCV) strain 229E (ATCC VR-740) and Betacoronavirus 1, strain OC43 (ATCC® VR-1558™) were obtained from the ATCC. HCV 229E is able to grow in human cell lines such as MRC-5 and produces CPE consisting of rounding and sloughing of cells. MRCS (ATCC CCL-171) is a human lung fibroblastic cell line obtained from a normal 14-week old male fetus. It supports the replication of a number of respiratory viruses including human coronaviruses. MRC-5 cells, 80-90% confluent, was infected at a relatively high multiple-of-infection (MOI of 0.1 to 0.2) and the virus was harvested 24-48 hours post infection before CPE is visible. HCV OC43 shows no cross reactivity with HCV strain 229E, and is able to grow in human cell lines such as HCT-8 (ATCC CCL-244) and produces CPE consisting of vacuolation and sloughing of cells. Mouse hepatitis virus strain MHV-A59, a mouse coronavirus, was also obtained from ATCC and grown in NCTC clone 1469, a mouse liver cell line, in which it produces CPE consisting of syncytia, rounding and sloughing of cells. The novel human coronavirus SARS-CoV-2, the causative agent of COVID-19, was grown in Vero E6 fetal rhesus monkey kidney cells; a plaque assay was used to titer the stock per established protocols.

Prototypical Enveloped and Nonenveloped Viruses. Virus stocks were produced, and virus titrations were performed following standard procedures established in the Aphios lab. BVDV (NADL strain, ATCC VR-1422) and HAd-2 (ATCC VR-846) virus stocks as well as their respective cell lines—BT cells (ATCC CRL-1390) and A549 (ATCC CCL-185), for virus culturing and titration were purchased from American Type Culture Collection (ATCC) and scaled-up in our BSL-3 facility. Virus culture was performed by infecting the monolayers at the optimal multiplicity of infection (m.o.i.) and harvesting the virus when cytopathic effects (CPE) are complete. Cell-free virus present in the culture supernatant and cell-associated virus were harvested separately but only cell-free virus stocks were used in the experiments since the total yields of cell-associated virus stocks are relatively low. Stock viruses were titrated on the respective host monolayers by infectivity titration. Cells were grown in 96-well plates and infected with serial log or half-log dilutions of virus stocks in replicates of 8 per dilution. When the CPE is complete (in 1-2 weeks), the number of wells showing CPE at each dilution was counted and the TCID₅₀ calculated by the Karber method.

The virus stocks generated above were titrated in 96 well plates by our standard TCID₅₀ procedure on their respective host cells. Briefly, confluent monolayers of the host cells were infected with serial log dilutions of the virus in replicates of 8. CPE was monitored for 5-10 days and the number of wells showing CPE was used to calculate the TCID₅₀ by the Karber method. The duration of the assay that gives the highest titers were optimized initially. Additionally, virus titrations are performed by qPCR of viral nucleic acids and ELISA and/or lateral flow assays for viral antigens in the culture supernatants in the TCID₅₀ assay.

Cytotoxicity and interference studies were performed in parallel with the efficacy studies by the various combinations of the four-component systems to ensure that any efficacy observed is not due to the effects of these compounds on the host cells. Cytotoxicity studies were performed by treating the cells with different doses of the combinations of drugs for the same durations as the efficacy assays. At the end of the treatment periods, the cells were visually examined for morphological changes and the metabolic activity assayed by a metabolic assay such as the CellTiter 96® AQueous One by Promega. The non-toxic doses for each of the combinations were determined by this method.

Bacterial Cultures. Stock cultures of E. coli and B. subtilis were obtained from ATCC and larger stocks prepared using the respective recommended bacteriological liquid media. The cultures were titrated as CFU/mL on the respective recommended media agar plates. The titered stock cultures were used in inactivation studies as described above for virus stocks, and the residual bacterial titers were determined on the respective recommended agar plates. Interference experiments were conducted to determine the effect of any leached compounds on bacterial titrations.

FFR Integrity. Decontamination might cause poorer fit, reduced filtration efficiency, and reduced breathability of disposable FFRs as a result of changes to the filtering material, straps, nose bridge material, or strap attachments of the FFR. Decontamination may produce chemical inhalation risks and should be evaluated for off-gassing.

A qualitative FFR performance evaluation was conducted as follows: (I) The FFR wearer dons their previously used FFR (for reuse) or wear an FFR (extended use); (2) The wearer dons the test hood.; (3) The test agent is released within the hood (add more test agent every 30 seconds); and (4) the wearer performs 7 exercises for 15 seconds each: Breathe normally; Breathe deeply; Move head side to side; Move head up and down; Talk; Bend over at the waist; and Breathe normally (CDC, 2000).

Filtration efficiencies of N95 FFRs were measured using the NIOSH NaCl aerosol test method, and FDA required particulate filtration efficiency (PFE) and bacterial filtration efficiency (BFE) methods, and viral filtration efficiency (VFE) method. Triplicate samples of each sample were tested using each method. Both PFE and BFE tests were done using un-neutralized particles as per FDA guidance document. PFE was measured using 0.1 μm size polystyrene latex particles and BFE with ˜3.0 μm size particles containing Staphylococcus aureus bacteria. VFE was obtained using ˜3.0 μm size particles containing phiX 174 as the challenge virus and Escherichia coli as the host.

Statistical Analysis. Data were analyzed using a Student's two-tailed t-test or one-way analysis of variance (1-way ANOVA) for measured (parametric) data or a Mann-Whitney U test (M-W) or Kruskal-Wallis (K-W) test for scored (non-parametric) data.

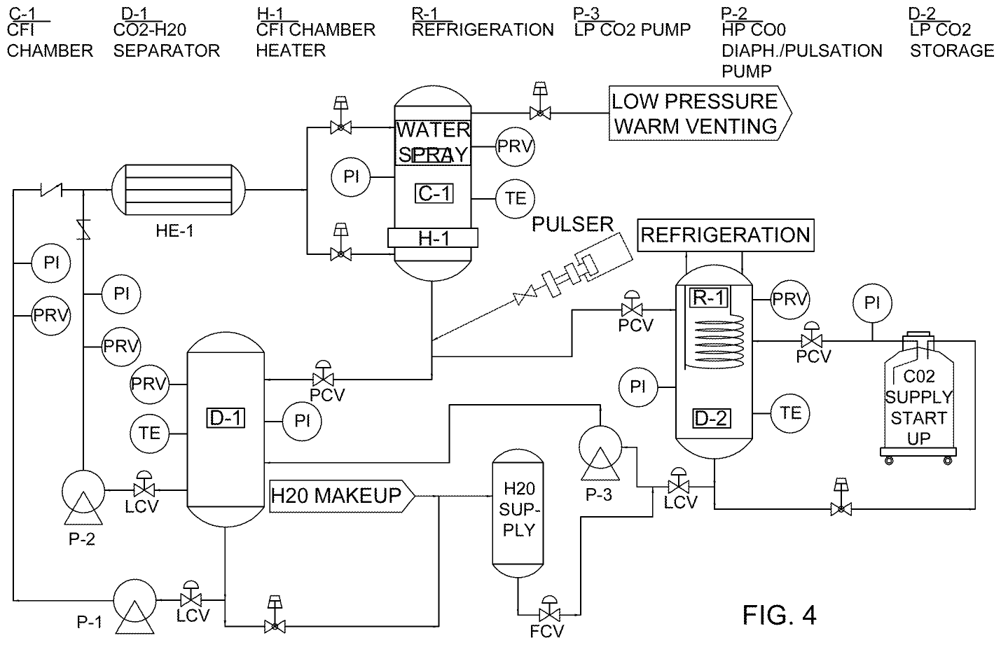

α-site CFI™ device for PPEs and SUDs that can operate following Good Laboratory Practice (GLP) procedures. We designed and constructed an α-site SuperFluids™ CFI™ Decontamination Device prototype. The design was based on experimental data developed based on prior research. The design of the α-site CFI™ prototype was modified to accommodate improvements and optimization of the CFI™ operating conditions. Based on results, several designs were evaluated. Some of these designs involved the use of refrigeration cycles; others eliminated the need for cosolvents and/or pressure cycling. All designs considered economic, operational and environmental considerations. A trade-off analysis was conducted with candidate CFI™ prototypes and conventional sterilization equipment. The design of the α-site CFI™ prototype was then be finalized based on the optimization studies conducted and trade-off analyses A preliminary CFI™ Decontamination Device prototype is shown as FIG. 4.

The heart of the α-site medical device prototype is a 100-liter CFI™ chamber rated for 3,000 psig. The CFI™ chamber has an automatic closure which can only be opened after the vessel is fully exhausted and the pressure in the chamber is zero or slightly negative. The CFI™ chamber contains a spray nozzle, such as a Bete-Fog or equivalent, for the introduction of trace quantities of water or a cosolvent such as ethanol into the SuperFluids™ stream. It is anticipated that the optimum concentration of water, if necessary, for the inactivation of coronaviruses and other prototypical viruses, were in the 1 to 3% range. It should be noted that trace quantities of water are necessary for ethylene oxide sterilization as well as the dry heat inactivation of tough nonenveloped viruses such as parvovirus. The chamber is heated so that its temperature can be maintained at an isothermal point ranging from room temperature (25° C.) to 60° C.

A pulsation device (pulser) is located on the exhaust line of the CFI™ chamber. The pulser is designed to generate pressure fluctuation in the CFI™ chamber for enhancing mixing between the SuperFluids™ and the PPEs and SUDs contaminated with coronaviruses and other potential pathogens. It is anticipated that the pressure pulsing or cycling will also increase the inactivation of viruses by disruption of envelope structures or protein capsids.

The exhaust from the CFI™ chamber is directed to a CO₂—H₂O separator (D-1), which operates around 800 psig. The water level is controlled by a level control valve (LCV), which directs water back to the H₂O supply tank or, as needed, to H₂O pump P-1 which recompresses the water from 800 psig to 3,000 psig and directs the pressurized water to the CO₂ recycle stream. The CO₂ from D-1 can be re-pressurized from 800 psig to 3,000 psig by high-pressure CO₂ pump P-2. The pressurized CO₂ and H₂O are mixed and then pre-heated in heat exchanger HE-1 before returned to the CFI™ chamber.

The low-pressure exhaust from the CFI™ chamber is directed to a low-pressure CO₂ storage tank D-2, which is refrigerated. The operating pressure and temperature of D-2 were maintained at 200 psig and −30° F. to maintain the CO₂ in a liquid state. As needed, low-pressure CO₂ is re-compressed up to 800 psig by the low-pressure CO₂ pump P-3 and directed to D-1. Any required water is added to this stream prior to re-compression from the H₂O supply tank. Small quantities of water makeup were added to the H₂O supply tank to maintain a constant supply level. A refrigerated liquid CO₂ tank, similar to the 600-liter liquid N2 tanks utilized in most health-care operations, was utilized for CO₂ supply startup and level maintenance in D-2.

After draining the CO₂/H₂O mixture in the CFI™ chamber down to 200 psig, the CFI™ chamber is vented to the atmosphere. During the venting process, the chamber is maintained at a warm temperature (>4° C.) in order to prevent freezing. Alternatively, the low-pressure CO₂ was pulled out under vacuum, recompressed to 200 psig and directed to low-pressure tank D-2. This alternative was utilized if venting is unacceptable to the regulatory authorities and/or if the SuperFluids™ of choice become a more expensive and potential environmental-sensitive alternative such as a fluorocarbon.

As an α-site unit, and based on preliminary but non-optimized operating conditions, the prototype is designed to be inherently flexible in terms of degrees of freedom and operating conditions. Operationally, the process steps entail: (1) medical devices loading: (2) carbon dioxide/water spray loading (3,000 psig at 31° C. to 40° C.); (3) pulsation/pressure fluctuation; (4) pressure letdown from 3,000 psig to 800 psig at 15° C.; (5) pressure letdown from 800 psig to 200 psig at −35° C.; (6) warm venting; and (7) unloading of medical devices. The process cycle is established at 60 minutes, with an additional 60 minutes required for loading and pressurization, and depressurization and unloading.

The unit is designed to operate under cGLP conditions with Clean-In-Place and Sterilization-In-Place features. Significant attention is paid to the pressure letdown system, and a no-fault interlocking system to prevent CFI™ device opening when under pressure greater than atmospheric pressure. The unit has redundant electrical and mechanical systems. The footprint of the unit is sized to accommodate placement in a hospital or medical institution.

Validation of the α-site CFI™ device for the inactivation of coronaviruses including SARS-CoV-2 on PPEs such as gowns, masks and face shields. The α-site CFI™ prototype is operated with several different simulated and actual PPEs loaded with different types of coronaviruses and other potential pathogens. The unit is tested in single and multiple cycles with different load conditions to evaluate its virucidal efficacy and operational robustness. Based on these tests, best operating conditions of pressure, temperature and time as well as operating parameters ranges are established for the selected SuperFluids™. The α-site CFI™ prototype is then validated by running three back-to-back batches, and evaluating the ability of the unit to maintain operating conditions and inactivate different viruses including coronaviruses.

The β-Site SuperFluids™ CFI™ Decontamination Device is designed based on the results of the α-site unit and used to perform technical and economic feasibility analyses. From a technical perspective, this evaluation encompassed potential efficacy against different strains of coronavirus and other viruses, operation cycle time and medical device volume turnover, ease of operation for a technician or nurse practitioner, and potential risks in product quality and device operation. From an economic perspective, the evaluation include capital and operating (life cycle) costs, maintenance strategies and costs and insurance risks/offsets. Both evaluations are compared to other pathogen decontamination alternatives including steam, ethylene oxide, gamma irradiation and hydrogen peroxide gas plasma.

While this invention has been particularly shown and described with references to specific embodiments, it will be understood by those skilled in the art that various changes in form and details may be made therein without departing from the spirit and scope of the invention as defined by the following examples and appended claims.

### EXAMPLES

### Example 1: SuperFluids™ CFI Decontamination of Personal Protective Equipment

The objective of this experiment was to inactivate Bacteriophage Φ-6-spiked Personal Protective Equipment, (PPE) utilizing SFS 99:1::CO2:H2O at 2,200 psig 33° C. in dynamic mode on the modified CFN apparatus.

N95 respirators and Medical-Grade face masks were cut into 7/16″ circles, spiked with Bacteriophage Φ-6 at a concentration of 10⁶ PFU. Pseudomonas virus Φ-6 Bacteriophage is a circular, enveloped bacteriophage with double-stranded RNA. Bacteriophage Φ-6 was used as a surrogate for coronavirus SARS-CoV-2 virus

Critical Fluid Inactivation was performed utilizing a modified Critical Fluid Nanosomes apparatus equipped with a 10 mL solids chamber and high-pressure circulation pump. The experiment consisted of (2) 30-min circulation cycles, one for each type of mask.

TABLE 2
Materials and Equipment:

| | |
|---|---|
| (Liquid) CO₂ tank and (Liquid) N₂O tank, both | Extech Thermocouple |
| with dip tubes, obtained from Specialty Gases | |
| of America | |
| One (1) Isco Syringe Pumps Model 260D; One | Soap Water |
| (1) Isco Syringe Pump Model 500HP; Pump | |
| Controller (S/N: 220E00013) | |
| Deareated DI Water | Modified CFN Apparatus |
| VWR Recirculating Bath Model # 1162 (used | Torque Driver |
| for chilling SFS pumps) | |
| Clorox | |

The results of the CFI pathogen reduction of PPEs' spiked with Bacteriophage Φ-6 are listed in Table 3.

TABLE 3
SuperFluids ™ CFI Pathogen Reduction of PPEs' Spiked with Bacteriophage Φ-6

| | | Number | | | Titer | |
|---|---|---|---|---|---|---|
| CFIU-V-01 | Dilution | of | No. in | Titer | (log | VRF |
| Sample | (log) | plaques | Undiluted | (pfu/mL) | pfu/mL) | (log pfu) |
| 8 log stock | 3 | TM | TM | 8.40E+07 | 7.92 | N/A |
| | 4 | 103* | TM | | | |
| | 5 | 42 | 4200000 | | | |
| Surgical - | U | TM | TM | 2.40E+05 | 5.38 | 0.00 |
| 4° C. control | 1 | TM | TM | | | |
| | 2 | 120 | 12000 | | | |
| | 3 | 5 | 5000* | | | |
| Surgical - | U | TM | TM | 4.00E+05 | 5.60 | −0.22 |
| t&T control | 1 | TM | TM | | | |
| | 2 | 160 | 16000 | | | |
| | 3 | 24 | 24000 | | | |
| Surgical - | U | 2 | 2 | 4.00E+01 | 1.60 | 3.78 |
| CFIU sample | 1 | Zero | Zero | | | |
| | 2 | Zero | Zero | | | |
| | 3 | Zero | Zero | | | |
| N95 - 4° C. | U | TM | TM | 2.69E+05 | 5.43 | 0.00 |
| control | 1 | TM | TM | | | |
| | 2 | 139 | 13900 | | | |
| | 3 | 13 | 13000 | | | |
| N95 - t&T | U | TM | TM | 2.73E+05 | 5.44 | −0.01 |
| control | 1 | TM | TM | | | |
| | 2 | 173 | 17300 | | | |
| | 3 | 10 | 10000 | | | |
| N95 - CFIU | U | 1 | 1 | <2.00E+01 | >1.30 | >4.13 |
| sample | 1 | Zero | Zero | | | |
| | 2 | Zero | Zero | | | |
| | 3 | Zero | Zero | | | |

*These numbers were considered outliers and not used for calculations.

The results showed 3.78 log reduction of Bacteriophage Φ-6 in surgical mask samples and >4.13 log reduction of Bacteriophage Φ-6 in N95 mask samples.

### Example 2: SuperFluids™ CFI Decontamination of Medical Devices

Virus preparation and SuperFluids™ CFI operating conditions for the medical device experiments are summarized in Table 4, and the results of medical device (MDV) experiments are summarized in Table 5.

TABLE 4
Conditions for Medical Device Experiments

| | Method of | Virus stock | p24 | Log | | | Pressure | Temp | Dynamic/ | Time |
|---|---|---|---|---|---|---|---|---|---|---|
| Exp. | drying | used | (ng/ml) | TCID₅₀/ml | SCF | Co-solvent | (psig) | (° C.) | Static | (min) |
| POC-01 | Air | Passage 1 | 68.8 | 1.9 | None | None | NA | NA | NA | NA |
| POC-02 | Lyophilized | Passage1 | 68.8 | 1.9 | None | None | NA | NA | NA | NA |
| MDV-01 | NA | None | NA | NA | CO₂ | None | 2200 | 22 | Static | 10 |
| MDV-02 | Lyophilized | Passage1 | 68.8 | 1.9 | CO₂ | None | 2200 | 22 | Static | 60 |
| MDV-03 | Savant | Passage 1 | 902.2 | 3.34 | CO₂ | None | 2200 | 24 | Static | 60 |
| MDV-04 | Sav./sucrcse | Passage 1 | 902.2 | 3.34 | CO₂ | None | 2200 | 24 | Static | 60 |
| MDV-05 | Lyophilized | Passage 2 | 134.7 | 5 | N₂O | None | 2200 | 22 | Static | 60 |
| MDV-06 | Lyophilized | Passage 2 | 134.7 | 5 | CO₂ | 10% water | 2200 | 23 | Static | 60 |
| MDV-07 | Lyophilized | Passage 2 | 134.7 | 5 | CO₂ | 1% water | 2200 | 24 | Static | 60 |
| MDV-08 | Savant | Passage 2 | 368.2 | 4.5 | CO₂ | 10% Ethanol | 2200 | 32 | Dynamic | 60 |
| | | | | | | 30 min. | | | | |
| MDV-09 | Savant | Passage 2 | 102.95 | 4.2 | CO₂ | 1% water | 2200 | 25 | Static | 60 |
| | | (conc.) | | | | | | | | |
| MDV-10 | Savant | Passage 2 | 90,610 | 7.55 | CO₂ | 10% Ethanol | 2200 | 32 | Dynamic | 60 |
| | | (conc.) | | | | 30 min. | | | | |
| MDV-11 | Savant | Passage 2 | 90,610 | 7.55 | Fr-23 | None | 2200 | 32 | Static | 60 |
| | | (conc.) | | | | | | | | |
| MDV-12 | Savant | MDV-10 & 11 | ND | ND | CO₂ | 1% water | 2200 | 33 | Static | 60 |
| MDV-13 | Savant | Passage 2 | IP | IP | Fr-22 | None | 2200 | 21 | Static | 60 |
| | | (conc.) | | | | | | | | |
| MDV-14 | Savant | Passage 2 | IP | IP | N₂O | 10% Ethanol | 2200 | 24 | Dynamic | 60 |
| | | (conc.) | | | | 30 min. | | | | |
| MDV-15 | Savant | Passage 2 | IP | IP | CO₂ | 1% water | 2200 | 23 | Static | 60 |
| | | (conc.) | | | | | | | | |

IP—In Progress
ND—Not Done
NA—Not Applicable

TABLE 5
Summary of Medical Device CFI Experiments

| | Log | Log | |
|---|---|---|---|
| | TCID₅₀/mL | TCID₅₀/mL | Log Kill |
| Exp. | control | treated | TCID₅₀/mL |
| MDV-02 | NA | NA | NA |
| MDV-03 | 2.45 | 1.7 | 0.75 |
| MDV-04 | 1.3 | 1.7 | −0.4 |
| MDV-05 | 2.1 | ND | >2.1 |
| MDV-06 | <0.7 | <1.7 | NA |
| MDV-07 | 2.45 | <1.7 | >1.7 |
| MDV-08 | 1.1 | <0.7 | >1.7 |
| MDV-09 | ND | ND | NA |
| MDV-10 | 3.45 | ND | >3.45 |
| MDV-11 | 4.2 | ND | >4.2 |
| MDV-12 | 3.75 | ND | 3.2 |
| MDV-13 | 4.3 | ND | >4.3 |
| MDV-14 | 4.2 | ND | >4.2 |
| MDV-15 | NA | NA | NA |

NA—Not Applicable
ND—Not Detected

The best results were obtained in MDV-13 with Freon-22 at 2,200 psig and 21° C. which inactivated >4.3 logs HIV-1; MDV-11 with Freon-23 at 2,200 psig and 32° C. which inactivated >4.2 logs HIV-1; MDV-14 with N20 and 10% ethanol at 2,200 psig and 24° C. which inactivated >4.2 logs HIV-1; and MDV-12 with CO2 and 1% water at 2,200 psig and 33° C. which inactivated 3.2 logs HIV-1

What we claim is:

1. A method for decontaminating medical equipment including personal protective equipment PPE, face fitting respiratory devices (FFR), and single-use medical devices (SUD) comprising the steps of: (a) placing the contaminated PPE in an isobaric processing chamber; (b) closing the chamber and introducing a SuperFluids into the chamber, said SuperFluids comprising a supercritical fluid, near critical fluid or critical fluid with or without a polar cosolvent at a predetermined temperature and pressure into the chamber; (c) keeping the SuperFluids in the chamber for a specified period of time sufficient to inactivate pathogens and viruses; and (d) removing the decontaminated PPE after processing.

2. The method of claim 1 wherein the supercritical, near-critical or critical fluid is carbon dioxide, nitrous oxide, propane and other alkanes and fluorocarbons.

3. The method of claim 2 wherein the preferred supercritical, near-critical or critical fluid is carbon dioxide.

4. The method of claim 1 wherein the polar cosolvent is water, acetone, methanol, and ethanol.

5. The method of claim 4 wherein the preferred polar cosolvent is water.

6. The method of claim 1 wherein the SuperFluids are at pressures ranging from 1,000 to 5,000 psig.

7. The method of claim 1 wherein the SuperFluids are at temperatures ranging from 20 to 60° C.

8. The method of claim 1 wherein the SuperFluids are a mixture of carbon dioxide and water with ratios ranging from 90% to 99% carbon dioxide and 10% to 1% water.

9. The method of claim 8 wherein the SuperFluids are a mixture of carbon dioxide and water with a ratio of 99% carbon dioxide and 1% water.

10. The method of claim 1 wherein the SuperFluids are a mixture of carbon dioxide and nitrous oxide.

11. The method of claim 1 wherein the SuperFluids are a mixture of carbon dioxide and nitrous oxide and a fluorocarbon.

12. The method of claim 1 wherein the SuperFluids are sonicated.

13. A method for decontaminating medical equipment including personal protective equipment PPE, face fitting respiratory devices (FFR), and single-use medical devices (SUD) comprising the steps of: (a) placing the contaminated PPE in an isobaric processing chamber; (b) closing the chamber and introducing a SuperFluids into the chamber, said SuperFluids comprising a supercritical fluid, near critical fluid or critical fluid with or without a polar cosolvent at a predetermined temperature and pressure into the chamber; (c) flowing the SuperFluids over the medical equipment in the chamber for a specified period of time sufficient to inactivate pathogens and viruses; and (d) removing the decontaminated PPE after processing.

14. The method of claim 1 wherein the supercritical, near-critical or critical fluid is carbon dioxide, nitrous oxide, propane and other alkanes and fluorocarbons and wherein the polar cosolvent is water, acetone, methanol, and ethanol.

15. The method of claim 13 wherein the preferred supercritical, near-critical or critical fluid is carbon dioxide the preferred polar cosolvent is water.

16. The method of claim 13 wherein the SuperFluids are at pressures ranging from 1,000 to 5,000 psig, and at temperatures ranging from 20 to 60° C.

17. The method of claim 13 wherein the SuperFluids are a mixture of carbon dioxide and water with a ratio of 99% carbon dioxide and 1% water.

18. The method of claim 13 wherein the SuperFluids are a mixture of carbon dioxide and nitrous oxide and a fluorocarbon.

19. The method of claim 13 wherein the SuperFluids are sonicated.

20. An apparatus for decontaminating personal protective equipment PPE, face fitting respiratory devices (FFR), and single-use medical devices (SUD) comprising: (a) a high pressure chamber with an automatic closure; (b) a spray nozzle for the introduction of trace quantities of water or a cosolvent such as ethanol into the SuperFluids™ stream; (c) The chamber is heated so that its temperature can be maintained at an isothermal point ranging from room temperature (25° C.) to 60° C.; (d) a pulsation device (pulser) on the exhaust line of the CFI™ chamber for enhancing mixing between the SuperFluids™ and the medical devices; (e) the exhaust from the CFI™ chamber is directed to a CO₂—H₂O separator; (f) the pressurized CO₂ and H₂O are mixed and then pre-heated in heat exchanger before returned to the CFI™ chamber; (g) the low-pressure exhaust from the CFI™ chamber is directed to a low-pressure CO₂ storage tank, which is refrigerated; (h) after draining the CO₂H₂O mixture in the CFI™ chamber, the CFI™ chamber is vented to the atmosphere and maintained at a warm temperature (>4° C.) in order to prevent freezing.
